(19) United States
(12) Patent Application Publication
(10) Pub. No.: US 2006/0282138 A1
(43) Pub. Date: Dec. 14, 2006

(54) Exothermic structure that is directly applied to skin

(75) Inventor: Keizo Ota, Fukui (JP)

Correspondence Address: OSHA LIANG L.L.P., 1221 MCKINNEY STREET, SUITE 2800, HOUSTON, TX, 77010, US

(73) Assignee: Ohshin MLP Co., Ltd., Takefu-shi (JP)

(21) Appl. No.: 11/149,936

(22) Filed: Jun. 10, 2005

(51) Int. Cl. A61F 7/12

(52) U.S. Cl. 607/96

(57) ABSTRACT

An exothermic structure that is directly applied to skin comprising an air-permeable sheet layer, an exothermic composition layer, an air-impermeable sheet layer, a layer of a non-woven fabric, and an adhesive layer in this order wherein the exothermic composition layer is put into a pouch that is made of the air-permeable sheet layer and the air-impermeable sheet layer is provided, wherein the adhesive layer is partly made on the layer of the non-woven fabric and the adhesive force of the adhesive layer is within the range of 2.80 to 8.50 newton when the adhesive force is determined by the method that is based on the examination method of D-935 (Examination for Adhesive Force of Adhesive Tape) in the Japan Pharmacopeia. This exothermic structure gives less tingle when it is peeled off although it has a necessary adhesive force.

### TECHNICAL FIELD

The present invention relates to an exothermic structure that is used by directly applying to skin.

### BACKGROUND ART

An exothermic structure of a disposable type, so-called a disposable body warmer, has been known as a device for simply warming up a part of human body. Among those exothermic structures, some ones are used by directly sticking on underware or by directly applying to skin. Especially for the latter one, various proposals, for example, following ones, have been given to prevent the exothermic structure from peeling off during its use and to prevent an occurrence of dermatitis such as a rubor, a rash, and the like even after a long-term use.

In a thermal structure that can absorb sweat, which is disclosed in Japanese Patent No. 2826667, one side of a pouch into which an exothermic composition is put has a three-layered structure comprising a layer of a synthetic resin film, a water-absorbable layer, and a layer of a porous synthetic resin film. At the outside of the layer of the porous synthetic resin film, a pressure-sensitive adhesive layer is partly made in the form of check or stripe. Because the thermal structure has the above-disclosed structure, body fluids such as sweat, waste matter, and the like, which have come on a skin surface by heat, are absorbed into the water-absorbable layer by passing through pores of the layer of the porous synthetic resin film in places where there are no pressure-sensitive adhesives. Therefore, the skin surface is maintained in sanitary conditions and the thermal structure tightly contacts the skin.

In an exothermic sheet that is disclosed in Japanese Patent Early-publication No. Hei. 9-557, an adhesive layer by which the sheet is applied to skin has a form of dots each having an outer diameter of 3 to 5 mm and the total area of the dots is limited to be 30 to 70 % of the area of the back (the side on which the adhesive layer is made) of the sheet. Places of the exothermic sheet where the adhesive layer does not exist constitute a continuous non-adhesive portion. Therefore, if this exothermic sheet is used, breathing through skin and perspiration are not repressed and the sheet is not peeled off by sweat.

In an exothermic sheet that is disclosed in Japanese. Patent Early-publication No. 2000-139990, an outside of one side of a pouch into which an exothermic composition is put is made of a hydrophilic non-woven fabric and a layer of a hydrophobic adhesive is made outside the layer of the non-unwoven fabric in the form of stripe or dots while parts of the adhesive penetrate the layer of the non-woven fabric. This exothermic sheet has portions where much heat is supplied (places where the adhesive exists) and other portions where less heat is supplied (other places where the adhesive does not exist). Therefore, accumulation of heat is restricted and thus fervescence or painful is prevented. Further, the hydrophilic non-woven fabric absorbs sweat and ejects it outside in the form of water vapor. Thus, also by this function, the effect that the accumulation of heat is prevented can be obtained, sweating due to moisture is prevented, and further the tendency of easy peeling off of the exothermic sheet due to sweat is prevented.

In a thermal structure for application that is disclosed in Japanese Patent Early-publication No. 2001-120588, one side of a pouch into which an exothermic composition is put is made of a laminated body comprising a hydrophilic layer that mainly comprises a hydrophilic fiber(s) and a hydrophobic layer that mainly comprises a hydrophobic fiber(s), and an adhesive layer is intermittently made outside the laminated body. Because the thermal structure has the above-disclosed structure, secretion such as sweat or the like is absorbed by the thermal structure.

### SUMMARY OF INVENTION

The present invention intends to provide an exothermic structure that is directly applied to skin, which structure gives less tingle when it is peeled off although it has a necessary adhesive force.

Further, the present invention intends to provide an exothermic structure that is directly applied to skin, which structure shows an excellent property about the absorption of fluids such as sweat and the like that human body secretes, and by which structure heat that has been generated from an exothermic composition can be suitably conducted to human body.

The present inventors have extremely studied to attain the above objects. As a result, they have accomplished the present invention.

Namely, the present invention provides an exothermic structure that is directly applied to skin comprising an air-permeable sheet layer, an exothermic composition layer, an air-impermeable sheet layer, a layer of a non-woven fabric, and an adhesive layer in this order wherein the exothermic composition layer is put into a pouch that is made of the air-permeable sheet layer and the air-impermeable sheet layer, characterized in that the adhesive layer is partly made on the layer of the non-woven fabric and the adhesive force of the adhesive layer is within the range of 2.80 to 8.50 newton when the adhesive force is determined by the following method that is based on the examination method of D-935 (Examination for Adhesive Force of Adhesive Tape) in the Japan Pharmacopeia:

(Method for Determining Adhesive Force of Adhesive Layer)

(1) one surface of an acrylic resin plate (9.5 cm or more×13 cm or more) is wiped with ethanol;
(2) an exothermic structure (9.5 cm×13 cm) is applied onto the acrylic resin plate in such a way that the side having a length of 9.5 cm of the exothermic structure is set to the side having a length of 9.5 cm or more of the acrylic resin plate;
(3) on the acrylic resin plate onto which the exothermic structure is applied wherein the exothermic structure lies below the acrylic resin plate, a weight of 800 g is put and they are left as they are at 37° C. for 30 minutes;
(4) the exothermic structure is turned up at an angle of 180 degrees at a half of its length (13 cm);
(5) a free end of the exothermic structure that has been arised by the turning up of the exothermic structure is hooked up to a tensile jig of an apparatus for a tensile test, and an end of the acrylic resin plate where the exothermic structure is not applied is held;
(6) by pulling the exothermic structure upward at a tensile rate of 50 mm per minute, the exothermic structure is peeled off from the acrylic resin plate and the force that is necessary for peeling off is determined; and
(7) the largest force in the step (6) is defined as the adhesive force of the adhesive layer of the exothermic structure.

The exothermic structure that is directly applied to skin includes embodiments wherein each embodiment has one member or two or more members among the following elements (A) to (F):
(A) the adhesive force of the adhesive layer is within the range of ball numbers 21 to 30 when the adhesive force is determined according to JIS Z 0237 (Examination Method for Adhesive Tape and Adhesive Sheet);
(B) the thickness of the adhesive layer is within the range of 20 to 100 μm;
(C) the total area of the adhesive layer is within the range of 20 to 70% of the total area of the surface for application;
(D) in each of the four sides of the surface for application, the ends of the adhesive layer exist at almost regular intervals;
(E) the non-woven fabric comprises a mixture of a hydrophilic fiber(s) and a hydrophobic fiber(s); and
(F) the exothermic structure further comprises a layer of a non-woven fabric outside the air-permeable sheet layer.

### BRIEF DESCRIPTION OF DRAWINGS

FIG. 1 is a schematic, sectional view of one example of the exothermic structure that is directly applied to skin according to the present invention.

FIG. 2 is a schematic, sectional view of another example of the exothermic structure that is directly applied to skin according to the present invention.

FIG. 3 is a schematic view of one example of the form of the adhesive layer in the exothermic structure that is directly applied to skin according to the present invention.

FIG. 4 is a schematic view of another example of the form of the adhesive layer in the exothermic structure that is directly applied to skin according to the present invention.

FIG. 5 is a schematic view of still another example of the form of the adhesive layer in the exothermic structure that is directly applied to skin according to the present invention.

FIG. 6 is a schematic view of still another example of the form of the adhesive layer in the exothermic structure that is directly applied to skin according to the present invention.

FIG. 7 is a schematic, perspective view to explain the method for determining the adhesive force of the adhesive layer.

### DETAIL DESCRIPTION OF INVENTION

Hereafter, the present invention will be particularly explained with referece to its preferable examples.

First, with reference to the figures, the physical constitution of the exothermic structure that is directly applied to skin of the present invention will be explained.

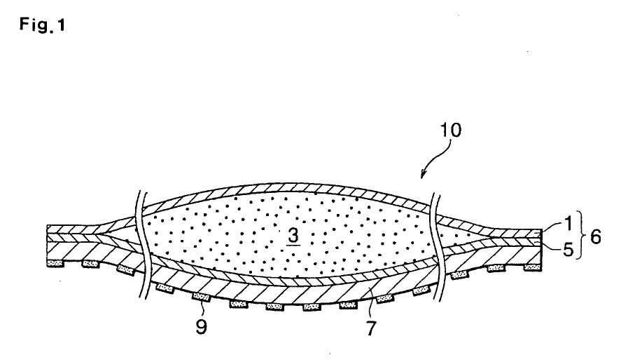

FIG. 1 is a schematic, sectional view of a preferable example of the exothermic structure that is directly applied to skin according to the present invention. The exothermic structure 10 that is directly applied to skin according to the present invention comprises an air-permeable sheet layer 1, an exothermic composition layer 3, an air-impermeable sheet layer 5, a layer 7 of a non-woven fabric, and an adhesive layer 9 in this order from the outside, and the exothermic composition layer 3 is put inside a pouch 6 that is made of the air-permeable sheet layer 1 and the air-impermeable sheet layer 5.

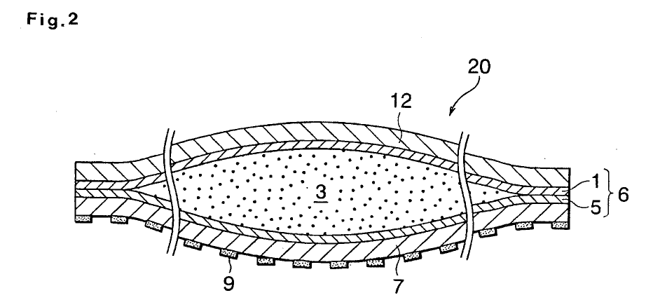

FIG. 2 is a schematic, sectional view of another preferable example of the exothermic structure that is directly applied to skin according to the present invention. The exothermic structure 20 that is directly applied to skin according to the present invention comprises a layer 12 of a non-woven fabric, an air-permeable sheet layer 1, an exothermic composition layer 3, an air-impermeable sheet layer 5, a layer 7 of a non-woven fabric, and an adhesive layer 9 in this order from the outside, and the exothermic composition layer 3 is put inside a pouch 6 that is made of the air-permeable sheet layer 1 and the air-impermeable sheet layer 5.

In the examples shown in FIGS. 1 and 2, the air-impermeable sheet layer 5 is adhered to the layer 7 of a non-woven fabric by heat-sealing. The air-impermeable sheet layer 5 may be adhered to the layer 7 of a non-woven fabric with an adhesive. In this case, an adhesive layer exists between the air-impermeable sheet layer 5 and the layer 7 of a non-woven fabric. The air-impermeable sheet layer 5 may be entirely or partly adhered to the layer 7 of a non-woven fabric.

In these examples, the periphery of the air-permeable sheet layer 1 is adhered to that of the air-impermeable sheet layer 5 by heat-sealing to constitute the pouch 6. The periphery of the air-permeable sheet layer 1 may also be adhered to that of the air-impermeable sheet layer 5 with an adhesive.

In the example showen in FIG. 2, the layer 12 of a non-woven fabric is partly bound to the air-permeable sheet layer 1 by heat-sealing or with an adhesive so that air reaches the exothermic composition layer 3 through these layers.

One of the characteristics of the exothermic structure that is directly applied to skin according to the present invention is that the adhesive layer is partly made on the layer of the non-woven fabric. Here, “partly made” means that there is at least one part where there is no adhesive layer on the layer of the non-woven fabric. The at least one part where there is no adhesive layer may be a continuous phase or non-continuous phases.

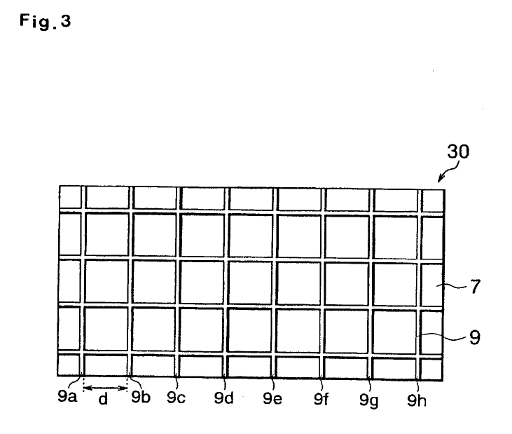

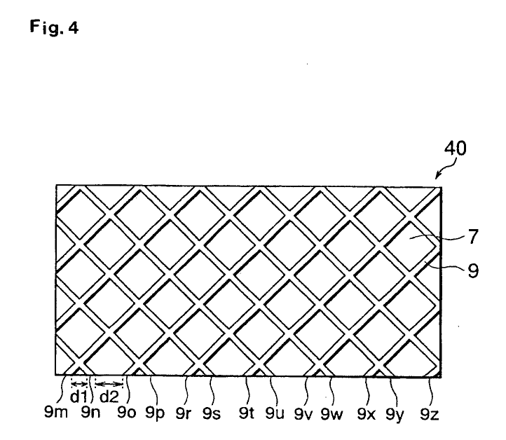

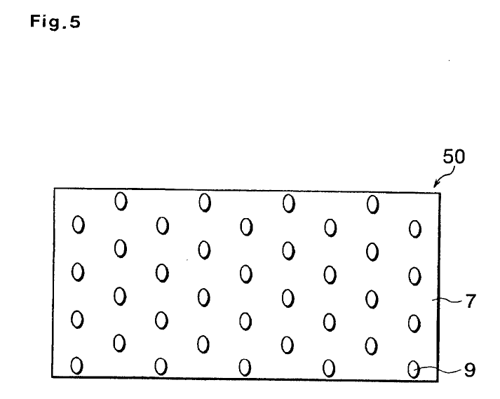

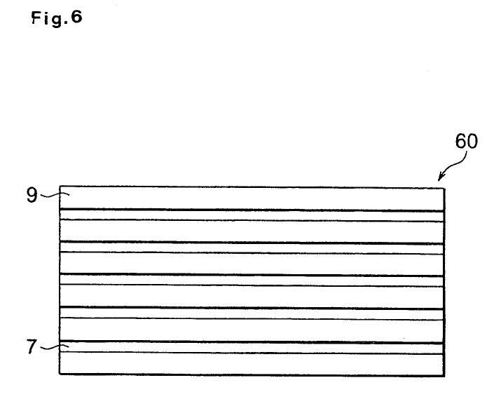

The adhesive layer is made in the form of, for example, check as shown in FIG. 3, biases that cross to one another as shown in FIG. 4, an aggregation consisiting of many dots as shown in FIG. 5, or a stripe as shown in FIG. 6.

Another one of the characteristics of the exothermic structure that is directly applied to skin according to the present invention is that the adhesive force of the adhesive layer is within the range of 2.80 to 8.50 newton when the adhesive force is determined by the following method that is based on the examination method of D-935 (Examination for Adhesive Force of Adhesive Tape) in the Japan Pharmacopeia. The adhesive force of the adhesive layer is within the range of preferably 2.80 to 6.00 newton, still more preferably 2.90 to 4.00 newton, and particularly preferably 3.00 to 3.50 newton.

(Method for Determining Adhesive Force of Adhesive Layer)

(1) One surface of an acrylic resin plate (9.5 cm or more×13 cm or more) is wiped with ethanol.

(2) An exothermic structure (9.5 cm×13 cm) is applied onto the acrylic resin plate in such a way that the side having a length of 9.5 cm of the exothermic structure is set to the side having a length of 9.5 cm or more of the acrylic resin plate.

(3) On the acrylic resin plate onto which the exothermic structure is applied herein the exothermic structure lies below the acrylic resin plate, a weight of 800 g is put and they are left as they are at 37° C. for 30 minutes.

(4) The exothermic structure is turned up at an angle of 180 degrees at a half of its length (13 cm).

(5) A free end of the exothermic structure that has been aeised by the turning up of the exothermic structure is hooked up to a tensile jig of an apparatus for a tensile test, and an end of the acrylic resin plate where the exothermic structure is not applied is held.

(6) By pulling the exothermic structure upward at a tensile rate of 50 mm per minute, the exothermic structure is peeled off from the acrylic resin plate and the force that is necessary for peeling off is determined.

(7) The largest force in the step 6 is defined as the adhesive force of the adhesive layer of the exothermic structure.

This method presumes that the adhesive force is determined by using an exothermic structure having a size of 9.5 cm×13 cm. The adhesive force is proportional to the width of the exothermic structure. Thus, if a determination was done by using an exothermic structure that does not have a width of 9.5 cm, an adhesive force in the case where the width is 9.5 cm should be calculated by reduction.

The adhesive force of the adhesive layer in the exothermic structure according to the present invention is within the range of preferably ball numbers 21 to 30, still more preferably ball numbers 22 to 30, and particularly preferably ball numbers 23 to 29, when the adhesive force is determined according to JIS Z 0237 (Examination Method for Adhesive Tape and Adhesive Sheet).

In the exothermic structure according to the present invention, it is preferable that the ends of the adhesive layer exist at almost regular intervals in each of the four sides of the surface for application. Here, “almost regular intervals” is a concept that includes not only the case where ends 9a, 9b, 9c, 9d, and the like of the adhesive layer 9 are set at regular intervals as shown in FIG. 3, but also the case where there are plural kinds of intervals that regularly occur although there are two or more sizes of the intervals between ends of the adhesive layer 9 (i.e., there are narrow intervals d1 such as the interval between ends 9m and 9n, the interval between ends 9o and 9p, and the like, and wide intervals d2 such as the interval between ends 9n and 9o, the interval between ends 9p and 9r, and the like) as shown in FIG. 4.

Although the total area of the adhesive layer is not particularly limited, it is within the range of, for example, 20 to 70%, preferably 20 to 60%, and particularly preferably 25 to 55%, of the total area of the surface for application (namely, one side of the exothermic structure).

Although the thickness of the adhesive layer is not particularly limited, it may be within the range of, for example, 20 to 100 μm, and is within the range of preferably 25 to 80 μm and particularly preferably 30 to 50 μm.

Next, materials and the like that respectively constitute the parts of the exothermic structure that is directly applied to skin according to the present invention will be specifically explained.

The exothermic composition layer of the exothermic structure that is directly applied to skin according to the present invention is constituted of an exothermic composition that generates heat by air. The components that are contained in this exothermic composition that generates heat by air are not particularly limited as long as they have been conventionally used in the exothermic compositions that generates heat by air. Examples of the components are as follows.

Examples of chemical exothermic agents include metal powders such as iron powders (reduced iron powder, atomized iron powder, and the like) and the like. Examples of reaction auxiliaries include metal halides such as sodium chloride, potassium chloride, magnesium chloride, calcium chloride, iron (II) chloride, iron (III) chloride, and the like; metal sulfates such as potassium sulfate, sodium sulfate, magnesium sulfate, copper sulfate, iron (II) sulfate, iron (III) sulfate, and the like; and the like. Examples of water retaining agents include active carbon, alumina, silica gel, zeolite, wood charcoal, water-absorptive polymeric compounds, and the like. Of course, water is also used. Examples of other additives include polymeric compounds such as carboxymethyl cellulose, acrylic acid starch, polyethylene, polypropylene, polystyrene, and the like; bentonite; vermiculite; pearlite; wood charcoal; and the like.

It is preferable that an exothermic composition that generate heat by air having a formula is used by which formula a metal powder such as an iron powder or the like has a less tendency of deflection.

The exothermic composition that generate heat by air is preferably processed so as to be a sheet-like form. In this case the thickness is within the range of preferably 5 mm or less, still more preferably 0.5 to 4 mm, and particularly preferably 1 to 2 mm.

In the exothermic structure that is directly applied to skin according to the present invention, the pouch into which the exothermic composition layer is put is constituted of an air-permeable sheet at one side and an air-impermeable sheet at the other side.

Examples of materials for the air-permeable sheet and the air-impermeable sheet that constitute the pouch include polyolefins such as polyethylene, polypropylene, and the like; polyamides such as nylons and the like; polyesters such as polyethylene terephthalate and the like; ethylene copolymers such as ethylene-vinyl acetate copolymers and their saponified ones, ethylene-alkyl (meth)acrylate copolymers, and the like; poly(vinyl chloride); poly(vinylidene chloride); polyurethanes; polystyrene; and the like. Also, natural rubbers, reclaimed rubbers, and synthetic rubbers may be used.

As a representative example of the air-permeable sheet, one obtained by making openings through which air passes in an air-impermeable polymer film (e.g., a moisture-permeable porous film) is cited. In this description, the term “moisture permeability” may be used. If moisture can pass through, a gas can also pass through. Namely, one having a moisture-permeability also has an air-permeability.

In the exothermic structure that is directly applied to skin according to the present invention, as shown in, for example, FIG. 2 a layer that is made of other material (the layer 12 of a non-woven fabric in the example of FIG. 2) may be laminated outside the air-permeable sheet layer 1. However, the layer that is made of other material and that is set outside the air-permeable sheet layer has to be one having an air-permeability. Examples of the other material that constitutes the layer that is set outside the air-permeable sheet layer include a woven fabric, a non-woven fabric, a knit, paper, and the like.

The air-permeability of the air-permeable side affects the exothermic property. Therefre, it is preferable to select and process a material(s) that constitutes the air-permeable side so that the air-permeable side that is made of only an air-permeable sheet layer or a composite comprising the air-permeable sheet layer and a layer made of other air-mermeable material has a moisture-permeability of 200 to 500 g/m²·24 hours (preferably 250 to 400 g/m²·24 hours) as represented by the moisture-permeability [JIS Z 0208 (1976)] that is determined by the Lyssy method.

Methods for processing materials that constitute the air-permeable side so that the air-permeable side has a desirable air-permeability, for example, a method for controlling the air-permeability through a bonding process when a laminate is used and a method for preparing a porous film having a desirable air-permeability, have been known.

If a moisture-permeable, porous film is used as the air-permeable sheet, its thickness is within the range of usually 100 μm or less, preferably 20 to 80 μm, and still more preferably 40 to 60 μm.

If the air-permeable layer that is set outside the air-permeable sheet layer is made of a woven fabric, a non-woven fabric, or paper, the thickness of the layer is, as represented by the basis weight, within the range of usually 200 g/m² or less, preferably 20 to 120 g/m², and still more preferably 40 to 100 g/m². If a non-woven fabric is used, a spun lace or spun bond non-woven fabric is preferred. Examples of materials of the non-woven fabric include rayons, nylons, polyesters, acrylics, polypropylene, Vinylon, polyethylene, urethanes, cotton, celluloses, and the like.

As representative examples of the air-impermeable sheet that constitutes another side (the air-impermeable side) of the pouch, air-impermeable polymer films, e.g., a polyethylene film, are cited. The thickness of the air-impermeable polymer film is within the range of usually 100 μm or less, preferably 10 to 70 μm, still more preferably 20 to 50 μm, and particularly preferably 25 to 45 μm.

Examples of the polymeric compound that constitutes the air-permeable sheet and the air-impermeable sheet include polyethylene, polypropylene, polyesters, polyamides, poly(vinyl chloride), poly(vinylidene chloride), polyurethanes, polystyrene, ethylene-vinyl acetate copolymers, polycarbonates, and the like.

The air-permeable sheet and the air-impermeable sheet are not limited to monolayer films, but may be multilayer films.

It is preferable that at least one of the air-permeable sheet and the air-impermeable sheet (with the proviso that if the sheet(s) is(are) a multilayer film(s), at least innermost layer(s) in these sheets) that constitute the pouch is a polymer film having a heat-sealability, e.g., metallocene polyethylene film.

In the exothermic structure that is directly applied to skin, the layer of the non-woven fabric that is set outside the air-impermeable sheet layer that constitutes one side (the side that faces skin) of the pouch plays a role of conveying heat that has been generated by the exothermic cimposition to human body, another role of absorbing liquids such as sweat and the like, and still another role as a support for maintaining the form of the exothermic structure.

The non-woven fabric that constitutes the layer of the non-woven fabric that is set outside the air-impermeable sheet layer is not particularly limited. However, it is preferably a spun lace or spun bond non-woven fabric. Among non-woven fabrics, those having a basis weight of 20 to 80 g/m² are preferable, those having a basis weight of 30 to 70 g/m² are still more preferable, and those having a basis weight of 40 to 50 g/m² are particularly preferable.

It is preferable that both a hydrophilic fiber and a hydrophobic fiber are used as the materials for the non-woven fabric. This is because the hydrophilic fiber is excellent in a property of absorbing liquids such as sweat and the like and the hydrophobic fiber is excellent in a heat-conductivity. Because the heat-conductivity of the layer of the non-woven fabric is reduced when the hydrophilic fiber absorbs a liquid such as sweat and the like, an effect is enhanced that a burn at low-temperatures is prevented. In consideration of these characteristics of the hydrophilic fiber and the hydrophobic fiber, a mixture comprising the hydrophilic fiber and the hydrophobic fiber at a ratio in the range of 10:90 to 70:30 (weight ratio) is preferably used, a mixture comprising those fibers at a ratio in the range of 20:80 to 70:30 (weight ratio) is more preferably used, and a mixture comprising those fiber at a ratio in the range of 30:70 to 50:50 (weight ratio) is particularly preferably used as the materials for the non-woven fabric.

Examples of the hydrophilic fiber include natural fibers such as cotton, wool, silk, hemp, wood pulp, and the like; cellulose fibers such as rayon, cupra, and th like; poly(vinyl alcohol) fibers; cellulose-acetate fibers; highly water-absorbable fibers (for example, crosslinked acrylate fibers, processed acrylic fibers of which surfaces are hydrolyzed, fibers that have been obtained by graft-polymerizing acrylic acid or methacrylic acid to fibers of polyesters and the like, etc.); and the like. Examples of the hydrophobic fiber include fibers that are made of polyesters, nylons, acrylics, and the like.

Examples of the adhesive composition that constitutes the adhesive layer that is partly made on the layer of the non-woven fabric include rubber-type adhesive compositions, acrylic-type adhesive compositions, other adhesive compositions each comprising as the main component a thermoplastic resin (for example, a polyaminde-type resin, a polyethylene-type resin, or a cellulose-type resin), and the like.

As the adhesives that are used in the rubber-type adhesive compositions, diene-type polymeric compounds, specifically natural rubber, synthetic rubbers, and mixtures of them are cited. As the synthetic rubbers, styrene-isoprene block copolymer rubber, styrene-isoprene-styrene block copolymer rubber, styrene-isobutylene-styrene block copolymer rubber, styrene-butadiene rubber, polyisoprene rubber, butyl rubber, chloroprene rubber, nitrile rubber, polysulfide rubber, silicone rubber, and the like are cited.

As the adhesives that are used in the acrylic-type adhesive compositions, conventionally-used copolymers of at least one (meth)acrylate such as n-butyl (meth)acrylate, hexyl (meth)acrylate, decyl (meth)acrylate, dodecyl (meth)acrylate, and tridecyl (meth)acrylate, with a functional monomer that is copolymerable with the (meth)acrylate such as (meth)acrylic acid, maleic acid, maleic anhydride, hydroxyethyl acrylate, hydroxypropyl acrylate, acrylamide, dimethylacrylamide, aminoethyl methacrylate, and methoxyethyl (meth)acrylate or a vinyl monomer that is copolymerable with the (meth)acrylate such as acrylonitrile, vinyl acetate, vinyl propionate, and the like, and the like are cited.

The condition of the adhesive composition that is used to make the adhesive layer is not partucularly limited as long as it can be readily applied onto the surface of the layer of the non-woven fabric, for example, an emulsion, a solution with a solvent, an aqueous solution, a hot-melt type one, etc.

In the exothermic structure that is directly applied to skin according to the present invention, before use the surface of the adhesive layer is covered with a releasable sheet. The materials for the releasable sheet are not limited as long as they have been conventionally used in the sheet for covering the adhesive layer of the exothermic structure, namely, in the releasable sheet. For example, various plastic films, metal foils, and a lamionate of a plastic film and paper are used as the releasable sheet. A coating agent for release, such as a silicone type, an alkylacrylate type, a fluorine type, or the like, may be applied onto the releasable sheet. Examples of polymeric compounds that constitute the plastic films include polyesters, polypropylene, polyethylene, alkylbenzene sulfonates, and poly(vinyl chloride).

The exothermic structure that is directly applied to skin according to the present invention (with the proviso that the surface of the adhesive layer is covered with a releasable sheet) is kept in an outer bag. The outer bag is constituted of a moisture resistant, air-impermeable material. Because the outer bag is air-impermeable, the exothermic agent in the exothermic composition that generates heat by air that constitutes the exothermic composition layer does not chemically react, and thus the exothermic composition is kept without generating heat. After the outer bag is opened, air (oxygen) gets to the exothermic agent through the pouch of the exothermic structure. Then, the chemical reaction starts and heat of reaction is emitted.

A representative example of the material for the outer bag is a laminate of aluminum foil with a polymer film.

Next, a process for preparing the exothermic structure that is directly applied to skin according to the present invention (with the proviso that the surface of the adhesive layer is covered with a releasable sheet and the exothermic structure is kept in an outer bag) will be explained.

In the case where the exothermic structure 10 as shown in FIG. 1 is produced, first, an air-permeable sheet (that will be the air-permeable sheet layer 1) and a sheet-like substance A wherein an air-impermeable sheet (that will be the air-impermeable sheet layer 5) is adhered to the side, on which side there is no adhesive layer 9, of a non-woven fabric (that will be the layer 7 of the non-woven fabric) having an adhesive layer 9 (which adhesive layer is covered with a releasable sheet that is not shown in the figure) that is partly made on one side of the non-woven fabric, are prepared. The preparation of the adhesive layer on one side of the non-woven fabric is conducted by using a coater of, e.g., an attached gravure system, a screen printing system, or the like. At almost the same time as the preparation of the adhesive layer, the surface of the adhesive layer is covered with a releasable sheet. The bonding between the non-woven fabric and the air-impermeable sheet is done, for example, by heat-sealing or with an adhesive. The bonding may be entirely or partly.

In the case where the exothermic structure 2 as shown in FIG. 2 is produced, instead of the air-permeable sheet (that will be the air-permeable sheet layer 1), a sheet-like substance B wherein an air-permeable is partly adhered to a non-woven fabric (that will be the layer 12 of the non-woven fabric) is used.

The air-permeable sheet (or the sheet-like substance B) and the sheet-like substance A are set so that the air-permeable sheet faces the air-impermeable sheet.

The air-permeable sheet (or the sheet-like substance B) is heat-sealed with the sheet-like substance A in a lateral direction. Next, their both sides are longitudinally heat-sealed to make a room. Into this room an exothermic composition that generate heat by air is put. Again, the sheet-like substances are laterally heat-sealed. Next, they are longitudinally heat-sealed to make a room in the same way as described above, and an exothermic composition that generate heat by air is put into the room. The room into which the exothermic composition that generates heat by air is put is pressed to have the exothermic composition that generate heat by air a form of a layer. Thereafter, the same operations are repeated so that continuous bodies into which the exothermic composition that generate heat by air is put are obtained.

Next, at the laterally heat-sealed part of the continuous bodies, cutting is done to separate one exothermic structure that is directly applied to skin (with the proviso that its adhesive layer is covered with a releasable sheet). This is put into an outer bag that is made of an oxygen-impermeable material. The cutting and the putting into the outer bag are repeated.

When the exothermic structure that is directly applied to skin according to the present invention is used, it is preferable that the exothermic structure is applied to skin while the releasable sheet is peeled off, after the exothermic structure has been get out from the outer bag.

Effect of Invention

The exothermic structure that is directly applied to skin according to the present invention shows effects that the structure gives less tingle when it is peeled off although it has a necessary adhesive force. When the structure is peeled off, the tingle comes about because, for example, hair that adheres to the adhesive layer of the exothermic structure is pulled or keratin of skin that adheres to the adhesive layer of the exothermic structure is peeled off.

Especially, the exothermic structure wherein the ends of the adhesive layer exist at almost regular intervals in each of the four sides of the surface for application among the exothermic structures that are directly applied to skin according to the present invention is hardly peeled off during its use.

The exothermic structure wherein the non-woven fabric comprises a mixture of a hydrophilic fiber(s) and a hydrophobic fiber(s) among the exothermic structures that are directly applied to skin according to the present invention also shows additional effects that the property about the absorption of fluids such as sweat and the like that human body secretes is excellent and that heat that has been generated from an exothermic composition can be suitably conducted to human body.

The exothermic structure which further comprises a layer of a non-woven fabric outside the air-permeable sheet layer among the exothermic structures that are directly applied to skin according to the present invention also shows an effect that feeling and property of maintaining its form are excellent.

### EXAMPLES

Hereafter, the present invention will be specifically explained with reference to the examples.

### Example 1

(Preparation of Exothermic Composition That Generates Heat by Air)

An exothermic composition that generates heat by air was prepared according to the formula shown in Table 1 by an ordinary method.

TABLE 1

| | Amounts |
|---|---|
| Names of Raw Materials | (wt. %) |
| Iron Powder | 60 |
| Active Carbon | 5 |
| Carboxymethyl Cellulose | 2 |
| Acrylic Acid Starch | 2 |
| Sodium Chloride | 2 |
| Ordinary Water | 29 |
| Total | 100 |

(Production of Exothermic Structure of Inventive Example)

An adhesive of a styrene-isoprene-styrene block copolymer type (manufactured by Nihon NSC; ME126) was applied onto a spun lace, non-woven fabric made of a polyester/rayon (80:20 by a weight ratio) (manufactured by Asahikasei; basis weight: 40 g/m²) in the form shown in FIG. 4 to prepare an adhesive layer. At the same time, this adhesive layer was covered with a film made of a polyester (one that is coated with a silicone; manufactured by Toyo Metallizing; Cerapeel; thickness: 38 μm).

The width, inetervals, and thickness of the adhesive layer are shown in Table 2.

A commercially available, air-impermeable polyethylene film (manufactured by Minacel; thickness: 40 μm) was adhered by heat-sealing to the non-woven fabric on which the adhesive layer is made. Thus, a sheet-like substance A was obtained.

A porous polyethylene film (manufactured by Kojin; TSF-EU; thickness: 50 μm) was partly adhered to a spun lace, non-woven fabric made of a polyester (manufactured by Asahikasei; basis weight: 60 g/m²). Thus, a sheet-like substance B was obtained. The moisture-permeability [JIS Z 0208 (1976)] of the sheet-like substance B when it was determined by the Lyssy method was 310 g/m²·24 hours.

The sheet-like substance A and the sheet-like substance B were laterally heat-sealed in the way that the air-impermeable polyethylene film faced the porous polyethylene film. Next, they were longitudinally heat-sealed to make a room, and 20 g of the exothermic composition that generates heat by air of which formula is shown in Table 1 was put into the room.

Again, these sheets were laterally heat-sealed to each other. The room into which the exothermic composition that generates heat by air is put was pressed to have the exothermic composition that generate heat by air a form of a layer having a thickness of about 1.5 mm. Thereafter, the same operations are repeated.

Cutting at the laterally heat-sealed part was done to separate one exothermic structure.

The size of the exothermic structure thus produced was 9.5 cm×13 cm. The width of the heat-sealed part at the periphery was 6 mm.

As exothermic structures of comparative examples, an exothermic structure (Comparative Example 1) having an adhesive layer all over the non-woven fabric that had been manufactured by M company and another exothermic structure (Comparative Example 2) having an adhesive layer in the form as shown in FIG. 6 that had been manufactured by H company were prepared. The sizes of them were also 9.5 cm×13 cm. The widths, inetervals, and thicknesses of the adhesive layers of the exothermic structures are shown in Table 2.

Examination Example 1

By the following method that is based on the examination method of D-935 (Examination for Adhesive Force of Adhesive Tape) in the Japan Pharmacopeia, the adhesive force of the adhesive layer of the exothermic structure was determined. Five samples per an exothermic structure were prepared, the adhesive forces of the adhesive layers of them were determined, and the averages were calculated. Table 2 shows the results.

(Method for Determining Adhesive Force of Adhesive Layer)

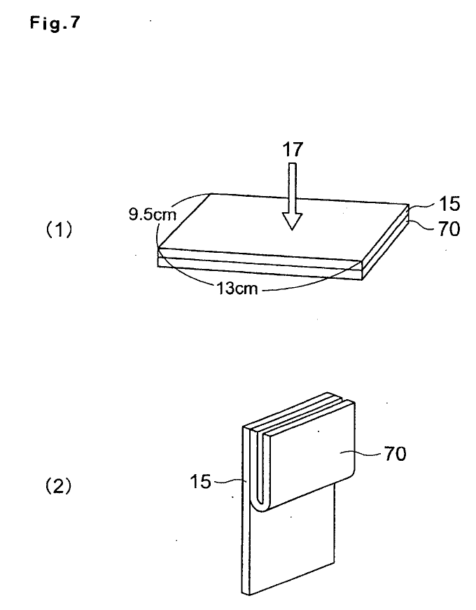

It will be explained with reference to FIG. 7.

(1) One surface of an acrylic resin plate 15 (9.5 cm×13 cm) is wiped with ethanol.

(2) An exothermic structure 70 is applied onto the acrylic resin plate 15 in such a way that the side having a length of 9.5 cm of the exothermic structure 70 is set to the side having a length of 9.5 cm of the acrylic resin plate 15.

(3) On the acrylic resin plate 15 onto which the exothermic structure 70 is applied wherein the exothermic structure 70 lies below the acrylic resin plate 15, a weight 17 of 800 g is put and they are left as they are at 37° C. for 30 minutes (see FIG. 7(1)).

(4) The exothermic structure 70 is turned up at an angle of 180 degrees at a half of its length (13 cm) (see FIG. 7(2)).

(5) A free end of the exothermic structure 70 that has been arised by the turning up of the exothermic structure 70 is hooked up to a tensile jig of an apparatus for a tensile test, and an end of the acrylic resin plate 15 where the exothermic structure 70 is not applied is held.

(6) By pulling the exothermic structure 70 upward at a tensile rate of 50 mm per minute, the exothermic structure 70 is peeled off from the acrylic resin plate 15 and the force that is necessary for peeling off is determined.

(7) The largest force in the step (6) is defined as the adhesive force of the adhesive layer of the exothermic structure 70.

TABLE 2

| | Inventive | Comparative | Comparative |
|---|---|---|---|
| | Example | Example 1 | Example 2 |
| Width of Adhesive Layer | 2 mm | — | 11 mm |
| Intervals of Adhesive Layer | 4.5 mm | — | 6 mm |
| Thickness of Adhesive Layer | 30 μm | 40 μm | 40 μm |
| Total Area of Adhesive Layer | 2,734 mm² | 12,350 mm² | 8,450 mm² |
| Ratio of Total Area of | 22% | 100% | 68% |
| Adhesive Layer to Total Area | | | |
| of Surface for Application | | | |
| Adhesive Force | 3.14 ± 0.26 | 11.17 ± 0.26 | 3.83 ± 0.25 |
| (Unit: newton) | | | |

Experimental Example 2

The adhesive force of the adhesive layer of the exothermic structure was determined according to JIS Z 0237 (Test Method for Adhesive Tape and Adhesive Sheet). Three samples per an exothermic structure were prepared, and the adhesive forces of the adhesive layers of them were determined. The results are shown in Table 3 as the ball numbers.

TABLE 3

| | Inventive | Comparative | Comparative |
|---|---|---|---|
| | Example | Example 1 | Example 2 |
| Sample 1 | No. 22 | No. 31 | No. 32 |
| Sample 2 | No. 21 | No. 32 | No. 32 |
| Sample 3 | No. 22 | No. 32 | No. 32 |

Experimental Example 3

Use test of the exothermic structures were performed.

Subjects were divided into to ten persons per one group. The details were that two subjects were thought that they were hirsure, three subjects were thought that they were somewhat hirsure, and five subjects thought that they had average hair.

Two different exothermic structures were applied onto right and left forearms of a subject, respectively. The subjects judged whether the exothermic structures were peeled off during use (for three hours) and the level of the tingle at removal.

The experiment was performed for three sets, namely, Inventive Example and Comparative Example 1, Inventive Example and Comparative Example 2, and Comparative Example 1 and Comparative Example 2.

Tables 4 to 6 shows the result.

TABLE 4
Comparison between Inventive Example and Comparative Example 1

| | | Inventive | | Comparative | |
|---|---|---|---|---|---|
| | | Example | | Example 1 | |
| Peeling Off | Peeled Off | 1 | person | 0 | person |
| During Use | Not Peeled Off | 9 | persons | 10 | persons |
| Tingle at | Tingled | 0 | person | 6 | persons |
| Removal | Tingled a Little | 3 | persons | 3 | persons |
| | Not Tingled | 7 | persons | 1 | person |

TABLE 4
Comparison between Inventive Example and Comparative Example 1

| | | Inventive | | Comparative | |
|---|---|---|---|---|---|
| | | Example | | Example 1 | |
| Peeling Off | Peeled Off | 1 | person | 0 | person |
| During Use | Not Peeled Off | 9 | persons | 10 | persons |
| Tingle at | Tingled | 0 | person | 3 | persons |
| Removal | Tingled a Little | 2 | persons | 4 | persons |
| | Not Tingled | 8 | persons | 3 | persons |

TABLE 4
Comparison between Comparative Examples 1 and 2

| | | Inventive | | Comparative | |
|---|---|---|---|---|---|
| | | Example | | Example 1 | |
| Peeling Off | Peeled Off | 0 | person | 0 | person |
| During Use | Not Peeled Off | 10 | persons | 10 | persons |
| Tingle at | Tingled | 5 | persons | 3 | persons |
| Removal | Tingled a Little | 5 | persons | 5 | persons |
| | Not Tingled | 0 | person | 2 | persons |

From the results shown in Tables 4 to 6, it is clear that when the exothermic structure of the present invention is used, during use the problem of peeling off hardly occurs and the tingle at removal is remarkably improved as compared to conventional ones (Comparative Examples 1 and 2).

Hereinbefore, the present invention is explained with reference to specific examples. However, the present invention is defined or limited only by the following claims.

What we claim are:

1. An exothermic structure that is directly applied to skin comprising an air-permeable sheet layer, an exothermic composition layer, an air-impermeable sheet layer, a layer of a non-woven fabric, and an adhesive layer in this order wherein the exothermic composition layer is put into a pouch that is made of the air-permeable sheet layer and the air-impermeable sheet layer, characterized in that the adhesive layer is partly made on the layer of the non-woven fabric and the adhesive force of the adhesive layer is within the range of 2.80 to 8.50 newton when the adhesive force is determined by the following method that is based on the examination method of D-935 (Examination for Adhesive Force of Adhesive Tape) in the Japan Pharmacopeia:
(Method for Determining Adhesive Force of Adhesive Layer)
(1) one surface of an acrylic resin plate (9.5 cm or more×13 cm or more) is wiped with ethanol;
(2) an exothermic structure (9.5 cm×13 cm) is applied onto the acrylic resin plate in such a way that the side having a length of 9.5 cm of the exothermic structure is set to the side having a length of 9.5 cm or more of the acrylic resin plate;
(3) on the acrylic resin plate onto which the exothermic structure is applied wherein the exothermic structure lies below the acrylic resin plate, a weight of 800 g is put and they are left as they are at 37° C. for 30 minutes;
(4) the exothermic structure is turned up at an angle of 180 degrees at a half of its length (13 cm);
(5) a free end of the exothermic structure that has been arised by the turning up of the exothermic structure is hooked up to a tensile jig of an apparatus for a tensile test, and an end of the acrylic resin plate where the exothermic structure is not applied is held;
(6) by pulling the exothermic structure upward at a tensile rate of 50 mm per minute, the exothermic structure is peeled off from the acrylic resin plate and the force that is necessary for peeling off is determined; and
(7) the largest force in the step (6) is defined as the adhesive force of the adhesive layer of the exothermic structure.

2. The exothermic structure that is directly applied to skin according to claim 1, wherein the adhesive force of the adhesive layer is within the range of ball numbers 21 to 30 when the adhesive force is determined according to JIS Z 0237 (Examination Method for Adhesive Tape and Adhesive Sheet).

3. The exothermic structure that is directly applied to skin according to claim 1, wherein the thickness of the adhesive layer is within the range of 20 to 100 μm.

4. The exothermic structure that is directly applied to skin according to claim 1, wherein the total area of the adhesive layer is within the range of 20 to 70% of the total area of the surface for application.

5. The exothermic structure that is directly applied to skin according to claim 1, wherein in each of the four sides of the surface for application, the ends of the adhesive layer exist at almost regular intervals.

6. The exothermic structure that is directly applied to skin according to claim 1, wherein the non-woven fabric comprises a mixture of a hydrophilic fiber(s) and a hydrophobic fiber(s).

7. The exothermic structure that is directly applied to skin according to claim 1, which further comprising a layer of a non-woven fabric outside the air-permeable sheet layer.
